(19) United States
(12) Patent Application Publication
(10) Pub. No.: US 2007/0077287 A1
(43) Pub. Date: Apr. 5, 2007

(54) Transdermal art patch

(76) Inventor: Corina S. Goodrich, Milwaukie, OR (US)

Correspondence Address: JAMES RAY & ASSOCIATES, 2640 PITCAIRN ROAD, MONROEVILLE, PA, 15148, US

(21) Appl. No.: 11/516,443

(22) Filed: Sep. 6, 2006

Related U.S. Application Data
(60) Provisional application No. 60/723,879, filed on Oct. 5, 2005.

(51) Int. Cl. A61K 9/70

(52) U.S. Cl. 424/449

(57) ABSTRACT

A transdermal patch having a pre-selected amount of a predetermined absorbable medicine thereon for attaching to an individual's skin. The transdermal patch includes a backing member having a predetermined size and a predetermined shape. The backing member has a first surface and a radially opposed second surface. A medicinal adhesive portion is disposed on the first surface for adhering to the individual's skin and administering the pre-selected amount of the predetermined absorbable medicine at a predetermined rate. A lining member is temporarily disposed over the first surface for providing a barrier between the medicinal adhesive portion and atmosphere until the transdermal patch is adhered to the individual's skin. A predetermined image portion is imprinted on the second surface for disguising an appearance of the transdermal patch.

### CROSS REFERENCE TO RELATED APPLICATION

This application is closely related to and claims benefit from U.S. Provisional Patent Application Ser. No. 60/723,879 filed on Oct. 5, 2005.

### FIELD OF THE INVENTION

The present invention relates, in general, to transdermal patches having a predetermined absorbable medicine thereon for attaching to an individual's skin and, more particularly, the invention relates to a medicinal transdermal patch having a predetermined image imprinted thereon such that the medicinal patch is disguised as artwork.

### BACKGROUND OF THE INVENTION

Prior to the conception and development of the present invention, as is generally well known in the prior art, individuals have been using transdermal patches for various health related issues for many years. Some common types of transdermal patches include nitroglycerin, hormone therapy, birth control, dieting, smoking cessation, etc.

Many patches currently available are recognizable by their uniform shape and size. These patches can be worn on various areas of the skin. However, if the individual is self-conscious about the patch being visible to others, then they are limited to certain areas of the skin and to certain types of clothing they can wear.

### SUMMARY OF THE INVENTION

The present invention provides a transdermal patch having a pre-selected amount of a predetermined absorbable medicine thereon for attaching to an individual's skin. The transdermal patch includes a backing member having a predetermined size and a predetermined shape. The backing member has a first surface and a radially opposed second surface. A medicinal adhesive portion is disposed on the first surface of the backing member for adhering to the individual's skin and administering the pre-selected amount of the predetermined absorbable medicine at a predetermined rate. A lining member is temporarily disposed over the first surface for providing a barrier between the medicinal adhesive portion and atmosphere until the transdermal patch is adhered to the individual's skin. A predetermined image portion is imprinted on the second surface of the backing member for disguising an appearance of the transdermal patch.

### OBJECTS OF THE INVENTION

It is, therefore, one of the primary objects of the present invention to provide a transdermal patch attachable to an individual's skin for administering an absorbable medicine into the individual.

Another object of the present invention is to provide a medicinal transdermal patch having an appearance different from patches currently available.

Still another object of the present invention is to provide a medicinal transdermal patch that is not recognizable as such when seen by other people.

Yet another object of the present invention is to provide a medicinal transdermal patch that can appear as a temporary tattoo or body art.

In addition to the above described objects and advantages of the present invention, various additional objects and advantages of the transdermal art patch according to the instant invention will become more readily apparent to those persons skilled in the relevant art from the following more detailed description, particularly, when such description is taken in conjunction with the attached drawing figures and with the appended claims.

### BRIEF DESCRIPTION OF THE DRAWINGS

FIG. 1 is a top view of a transdermal patch according to the present invention showing an artistic image imprinted thereon;

FIG. 2 is a schematic rear view of the transdermal patch, illustrated in FIG. 1, showing a lining member partially removed from the backing member revealing a medicinal adhesive portion; and

FIG. 3 is a schematic top view of the transdermal patch, illustrated in FIGS. 1 and 2, showing the lining member being pulled away from the backing member.

### DETAILED DESCRIPTION OF THE PRESENTLY PREFERRED AND VARIOUS ALTERNATIVE EMBODIMENTS OF THE INVENTION

Prior to proceeding to the more detailed description of the present invention it should be noted that, for the sake of clarity in understanding the invention, identical components having identical functions have been identified with identical reference numerals throughout the several views illustrated in the drawings.

The present invention offers a creative and attractive new look as an alternative to the recognizable appearance of currently available transdermal patches. The individual can wear the transdermal art patches anywhere they choose without being self-conscious about the patch being visible to others.

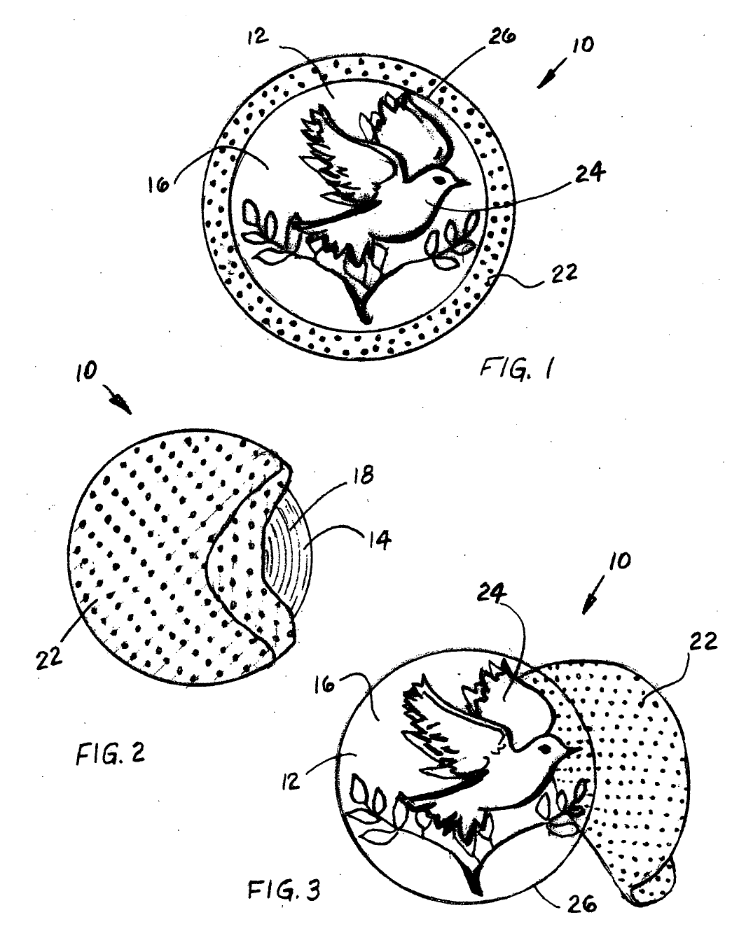

Now reference is made, more particularly, to the drawing FIGS. 1-3. Illustrated therein is a transdermal patch, generally designated 10, having a pre-selected amount of a predetermined absorbable medicine thereon for attaching to an individual's skin. The transdermal patch 10 includes a backing member 12 having a predetermined size and a predetermined shape. The backing member 12 has a first surface 14 and a radially opposed second surface 16.

The backing member 12 of the transdermal patch 10 will, preferably, be generally transparent. Also, the material used to make the backing member 12 will, preferably, be substantially impervious to liquids.

A medicinal adhesive portion 18 is disposed on the first surface 14 of the backing member 12 for adhering to the individual's skin and administering the pre-selected amount of the predetermined absorbable medicine at a predetermined rate. The predetermined absorbable medicine contained in the medicinal adhesive portion 18 of the transdermal patch 10 may be either prescription strength or nonprescription strength. For example, the predetermined absorbable medicine contained in the medicinal adhesive portion 18 may be selected from the group consisting of nitroglycerin, hormone therapy medications, birth control medications, dieting medications, pain control medications and smoking cessation medications.

A lining member 22 is temporarily disposed over the first surface 14 for providing a barrier between the medicinal adhesive portion 18 and the atmosphere until the transdermal patch 10 is adhered to such individual's skin.

A predetermined image portion 24 is imprinted on the second surface 16 of the backing member 12 for disguising an appearance of the transdermal patch 10. The predetermined image portion 24 imprinted on the second surface 16 of the backing member 12 may be selected from the group consisting of numerals, letters, artwork and various combinations thereof.

In one embodiment, the predetermined shape of the backing member 12 is one of a circular outline 26 and a square outline (not shown) of the predetermined image portion 24 imprinted thereon. In addition, the predetermined size of the backing member 12 is substantially identical to the dimensions of the predetermined image portion 24 imprinted thereon.

In another alternative embodiment of the invention, the transdermal patch 10 may include a portion disposed thereon having a predetermined scent.

While a presently preferred and various alternative embodiments of the present invention have been described in detail above it should be understood that various other embodiments of the invention can be envisioned by those persons skilled in the art without departing from the spirit of the invention or the scope of the appended claims.

I claim:

1. A transdermal patch having a pre-selected amount of a predetermined absorbable medicine thereon for attaching to an individual's skin, said transdermal patch comprising:
(a) a backing member having a predetermined size and a predetermined shape, said backing member including a first surface and a radially opposed second surface;
(b) a medicinal adhesive portion disposed on said first surface of said backing member for adhering to such individual's skin and administering said pre-selected amount of said predetermined absorbable medicine at a predetermined rate;
(c) a lining member temporarily disposed over said first surface for providing a barrier between said medicinal adhesive portion and atmosphere until said transdermal patch is adhered to such individual's skin; and
(d) a predetermined image portion imprinted on said second surface of said backing member for disguising an appearance of said transdermal patch.

2. A transdermal patch, according to claim 1, wherein said predetermined image portion imprinted on said second surface of said backing member is selected from the group consisting of numerals, letters, artwork and various combinations thereof.

3. A transdermal patch, according to claim 1, wherein said backing member is generally transparent.

4. A transdermal patch, according to claim 1, wherein said backing member is substantially impervious to liquids.

5. A transdermal patch, according to claim 1, wherein said predetermined shape of said backing member is one of a circular outline and a square outline of said predetermined image portion imprinted thereon.

6. A transdermal patch, according to claim 1, wherein said predetermined size of said backing member is substantially identical to dimensions of said predetermined image portion imprinted thereon.

7. A transdermal patch, according to claim 1, wherein said predetermined absorbable medicine contained in said medicinal adhesive portion of said transdermal patch is one of prescription strength and nonprescription strength.

8. A transdermal patch, according to claim 7, wherein said predetermined absorbable medicine contained in said medicinal adhesive portion of said transdermal patch is selected from the group consisting of nitroglycerin, hormone therapy medications, birth control medications, dieting medications, pain control medications and smoking cessation medications.

9. A transdermal patch, according to claim 1, wherein said transdermal patch includes a portion disposed thereon having a predetermined scent.
